(19) United States
(12) Patent Application Publication
(10) Pub. No.: US 2011/0009741 A1
(43) Pub. Date: Jan. 13, 2011

(54) Endovascular Optical Coherence Tomography Device

(75) Inventors: Marlon S. Matthews, Cheektowaga, NY (US); Jianping Su, Irvine, CA (US); Chiedozie I. Nwagwu, Newport Beach, CA (US); Zhongping Chen, Irvine, CA (US); Mark E. Linskey, Newport Beach, CA (US)

Correspondence Address: Law Offices of Daniel L. Dawes;Dawes Patent Law Group, 5200 Warner Blvd, Ste. 106, Huntington Beach, CA, 92649, US

(73) Assignee: THE REGENTS OF THE UNIVERSITY OF CALIFORNIA, Oakland, CA (US)

(21) Appl. No.: 12/863,949

(22) PCT Filed: Jan. 20, 2009
(86) PCT No.: PCT/US09/31465

§ 371 Date: Sep. 1, 2010

Related U.S. Application Data
(60) Provisional application No. 61/022,776, filed on Jan. 22, 2008.
(60) Provisional application No. 61/022,501, filed on Jan. 21, 2008.

(51) Int. Cl. A61B 6/00

(52) U.S. Cl. 600/425

(57) ABSTRACT

An endovascular OCT probe is included in an endovascular access device for intravascular imaging. The probe includes a hollow coil wire defining an axial lumen of the endovascular access device. A single mode optical fiber for transmitting light is disposed in the axial lumen of the hollow coil wire so that translation and rotation of the hollow coil wire carrying the optical fiber within the endovascular access device is stabilized for scanning endovascular tissue with at least 5 microns resolution. An optic element directs light from and into the optical fiber at a distal tip of the optical fiber and is coupled to or fixed to the distal end of the optical fiber. The optic element and the distal end of the optical fiber is disposed within a glass ferule to protect it from damage.

### RELATED APPLICATIONS

The present application is related to U.S. Provisional Patent Application, Ser. No. 61/022,776 filed on Jan. 22, 2008, and U.S. Provisional Patent Application, Ser. No. 61/022,501 filed on Jan. 21, 2008, which are incorporated herein by reference and to which priority is claimed pursuant to 35 USC 119.

### BACKGROUND OF THE INVENTION

1. Field of the invention

The invention relates to the field of endovascular OCT device incorporated in the context of an intracranial endovascular access device for intravascular imaging.

2. Description of the Prior Art

Optical coherence tomography (OCT) is a technology that permits high resolution in vivo imaging. Imaging is carried out at near histology resolution thus yielding clinically relevant information without the need for surgical biopsy. OCT has been used in vivo to image coronary artery disease. The in vivo clinical use of OCT has never been reported to study cerebral vasculature. To study cerebral, coronary, and other vasculature in the body such as in the kidneys, arms and legs as well as other organs OCT technology needs to be incorporated into an endovascular device that has all the necessary mechanical, physical and biological properties for proper device delivery and application.

The problem of obtaining in vivo blood vessel microstructure was previously studied using intravascular ultrasound (IVUS). OCT study has been previously reported in the coronary arteries, but never in the intracranial or other blood vessels outside the heart. The main disadvantage of IVUS is low resolution imaging. IVUS has not been used for in vivo imaging of intracranial blood vessels.

However, in order to obtain near histological resolution of which OCT is capable, it is necessary first to have some kind of OCT probe which can be used in endovascular applications, including intracranial applications, which can reproducibly and reliably deliver images having OCT quality resolution, which in turn requires stability at microscopic scales in the scanning mechanism and design of the OCT probe.

### BRIEF SUMMARY OF THE INVENTION

OCT is an imaging technique analogous to ultrasound except that IR or NIR light is used instead of sound. The optical beam is focused into the tissue and the reflection or echo time delay of the light which is scattered or reflected from internal microstructure in the tissue at different depths and resolved by interferometry. The image is obtained by performing repeated axial measurements at different transverse positions in the vessels as the optical beam is scanned across the tissue. The result is a two or three dimensional map of the reflectance from the internal morphology of the tissues and cells. Axial resolution of less than 5 microns can be obtained using relatively inexpensive and portable equipment, which is ideally suited for incorporation into catheters and endoscopes, which allow for optical biopsies at near histological levels without incision in a noninvasive manner.

The illustrated embodiment of the invention is an endovascular OCT probe included in an endovascular access device for intravascular imaging including a longitudinal lumen defined in the endovascular access device, a hollow coil wire defining an axial lumen therein, a single mode optical fiber for transmitting light, the fiber being disposed in the axial lumen of the hollow coil wire so that translation and rotation of the hollow coil wire carrying the optical fiber within the endovascular access device is stabilized for scanning endovascular tissue; and an optic element for directing light from and into the optical fiber at a distal tip of the optical fiber.

The endovascular access device has a proximal end and further comprises a FC/APC or FC/PC single mode fiber adaptor assembled thereto.

The endovascular access probe has a distal end which is used for imaging tissue, and where the optic element includes a GRIN lens and prism disposed on the distal end of the optical fiber to direct light in a beam perpendicular to the longitudinal axis of the fiber, and a glass ferrule encasing the GRIN lens and prism to protect the optic element and distal tip of the optical fiber from mechanical damage.

The endovascular OCT probe further includes a hollow steel tube disposed within the axial lumen of the hollow coil wire within which tube the optical fiber is disposed, the hollow steel tube being provided in at least a proximal of the probe whereby torsional stiffness of the OCT probe is increased to increase rotational and translational scanning stability of the OCT probe.

The endovascular OCT probe further comprises a fiber rotator and a linear stage transducer separately coupled to a proximal end of the OCT probe for provide for independently controlled rotational scanning and translational scanning of images.

The endovascular OCT probe further comprises an OCT interferometer and computer coupled thereto for generating images from the returned light from the optical fiber of the probe.

The endovascular OCT probe includes an annular space between the lumen of the endovascular access device and the hollow coil wire to allow for controlled flushing through a distal end of the OCT probe effective to flush a space between the optic element and tissue being scanned.

The endovascular OCT probe further comprises a hollow steel tube disposed within the axial lumen of the hollow coil wire within which tube the optical fiber is disposed, the hollow steel tube being provided only in a proximal portion of the probe whereby torsional stiffness of the OCT probe is increased to increase rotational and translational scanning stability of the OCT probe, and a second more distal hollow coil wire of smaller diameter than the proximal hollow coil, the second distal smaller coil wire including the optical fiber extending from the proximal coil wire and terminating in the optic element, the proximal hollow coil wire being characterized by greater stiffness than the distal hollow coil wire.

The endovascular OCT probe further comprises an external OCT system and electric motors coupled to the optical fiber and controlled by the external OCT system, wherein the OCT probe is separately rotated by a fiber rotator and/or linearly translated by a linear stage which are powered by the electric motors controlled by the external OCT system to generate a reproducible image with spatial resolution of at least 5 microns.

The proximal hollow coil wire is comprised of an outer coil made by removing the inner core of a larger diameter guide wire, and the distal hollow coil wire is comprised of a smaller coil made by removing the inner core of a smaller diameter guide wire.

The illustrated embodiment of the invention further includes an endovascular OCT probe for intravascular imaging comprising a microcatheter with a longitudinal lumen defined therein, a plurality of hollow coil wires coupled longitudinally to each other to collectively define a longitudinal lumen through the plurality of hollow coil wires, the plurality of hollow coil wires having a maximum stiffness and diameter at a proximal end of the plurality of hollow coil wires and sequencing monotonically with smaller diameters and less stiffness for each of the hollow coil wires until reaching a minimum stiffness and diameter at a distal end of the plurality of hollow coil wires, an optical fiber for transmitting light being disposed in the longitudinal lumen so that translation and rotation of the hollow coil wire carrying the optical fiber within the endovascular access device is stabilized for scanning endovascular tissue, and an optic element for directing light from and into the optical fiber at a distal tip of the optical fiber.

The illustrated embodiment also includes a method using a stable scanning endovascular OCT probe comprising the steps of endovascularly rotationally optically scanning tissue and/or endovascularly translationally optically scanning tissue with a stability able to achieve at least 5 micron resolution using the OCT probe in an intracranial or extracranial application, where endovascularly rotationally optically scanning tissue is performed in a torsionally reinforced endovascular OCT probe, and endovascularly translationally optically scanning tissue is performed in a longitudinally reinforced endovascular OCT probe.

The intracranial applications include assessment and evaluation of cerebrovascular diseases including but not restricted to atherosclerosis, fibromuscular dysplasia, inflammatory diseases of blood vessels, genetic, chromosomal, developmental, degenerative, and acquired abnormalities such as aneurysms, arteriovenous malformations, dissections, amyloid deposition, blood vessel ruptures and tears, evaluation of therapeutic manipulations such as aneurysm coiling, angioplasty, stent placement, AVM embolizations, tumor embolizations, assessment of therapeutic/healing response to surgical and medical treatments or prognostication of medical conditions involving blood vessels.

The extracranial applications are the same as intracranial applications but are carried out in blood vessels supplying blood to other organs such as the heart, liver, kidney, extremities, lungs, gastrointestinal tract including cerebral aneurysms by imaging the quantity and quality of fibrous connective tissue including collagen and elastin fibers within the aneurysm all to predict risk of rupture of cerebral aneurysms, or by imaging neo-endothelization across the neck of aneurysms treated with coils to document the extent of post-treatment aneurysm healing.

The method further comprises the step of flushing a bolus of 20 cc of normal saline into an endovascular space of interest through an endovascular catheter of appropriate size that is placed two to three centimeters proximal to the imaging field to flush away luminal blood, which otherwise obstructs the path of light during scanning.

The step of endovascularly rotationally optically scanning tissue and/or endovascularly translationally optically scanning tissue with a stability able to achieve at least 8 micron resolution using the OCT probe in an intracranial or extracranial application is solely motor controlled.

The step of endovascularly rotationally optically scanning tissue and/or endovascularly translationally optically scanning tissue with a stability able to achieve at least 5 micron resolution using the OCT probe in an intracranial or extracranial application is separately controlled and separately transduced rotation and translation of the OCT probe.

The step of endovascularly rotationally optically scanning tissue is performed in a torsionally reinforced endovascular OCT probe, and endovascularly translationally optically scanning tissue is performed in a longitudinally reinforced endovascular OCT probe comprised of a longitudinal lumen defined in an endovascular access device, a hollow coil wire defining an axial lumen therein, a single mode optical fiber for transmitting light, the fiber being disposed in the axial lumen of the hollow coil wire so that translation and rotation of the hollow coil wire carrying the optical fiber within the endovascular access device is stabilized for scanning endovascular tissue, and an optic element for directing light from and into the optical fiber at a distal tip of the optical fiber.

The step of endovascularly rotationally optically scanning tissue is performed in a torsionally reinforced endovascular OCT probe having an optical fiber disposed in the lumen of a hollow coil wire and an optic element fixed to a distal end of the optical fiber.

The step of endovascularly translationally optically scanning tissue is performed in a longitudinally reinforced endovascular OCT probe having an optical fiber disposed in the lumen of a thin solid hollow steel tube and an optic element fixed to a distal end of the optical fiber, the steel tube being disposed with a hollow coil wire.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

FIG. 1 is a side cross-sectional diagram of a first embodiment of the invention.

FIG. 2 is a side view of another embodiment of an OCT probe of the invention.

FIG. 3 is a side view of still another embodiment of an OCT probe of the invention.

FIG. 4 is a diagrammatic view of a rotator and linear stage transducer used to scan the OCT probe.

FIG. 5 is a diagram of an OCT interferometric system.

FIG. 6 is a side cross-sectional diagram of one application of the invention to image aneurysm necks occluded by GDC coils.

FIG. 7 is a photograph of the first purely translational scan of a human intracranial internal carotid artery wall taken with a probe of the invention.

FIG. 8 is a photograph of the first purely rotational scan of a human intracranial internal carotid artery wall and stent taken with a probe of the invention.

FIG. 9 is a side view of yet another embodiment of an OCT probe of the invention.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF THE PREFERRED EMBODIMENTS

The illustrated embodiment of the invention is an endovascular OCT device incorporated onto the framework of an intracranial endovascular access device for intravascular imaging. This construct is obtained by removing the solid core of an intracranial access wire while retaining the outer coiled shell. A single mode optical fiber is inserted into the hollow coil wire. In the proximal end that remains outside the body a FC/APC or FC/PC single mode fiber adaptor is assembled. In the distal end which is used for imaging tissue, a gradient-index (GRIN) lens and prism are glued together to focus and guide the light into a beam perpendicular to the fiber's longitudinal axis. A glass ferrule with strong medical glue inside protects the tip from mechanical damage. This construct confers the OCT device with adequate mechanical, biological, radiological, and optical properties necessary for in vivo endovascular imaging in patients. FC/PC and FC/APC connectors are most commonly found in high-end single mode fiber telecommunications systems. The term “FC” is a fiber connector designated by NTT. “PC” and “APC” describe the kind of polish applied to the connector end face. PC stands for physical contact. A PC connector has a polished convex end face. SPC and UPC are “super” polished and “ultra” polished with better back reflection specification than PC. APC stands for an angled physical contact. An APC connector has a polished end face angled at 8 degrees.

The purpose of the invention is to obtain high-resolution images of blood vessel wall to guide therapeutic decision-making and medical care of patients. OCT is analogous to ultrasound except that imaging is performed with light instead of acoustic waves. OCT measures light reflected from tissue structures. OCT is an imaging technique capable of performing high-resolution (3 to 8 microns), cross-sectional imaging. OCT enables real-time, in situ visualization of tissue microstructure without the need to excise and process the specimen as required for conventional biopsy and histopathology. Consequently, OCT is a powerful method to image biological tissue in-vivo at high resolution. OCT relies on scanning a region of interest to create images. Scanning requires rotational and translational movement which is accomplished by delivery of mechanical forces to the scanning tip of an OCT optical fiber. Consequently a safe, reliable, endovascular device is necessary for mechanical translation and rotation, as well as for delivery of light.

The main advantage of OCT over IVUS is its high-resolution imaging. The typical resolution using IVUS is 100 microns, while using OCT is 3 to 8 microns. Thus the resolution and quality of images obtained using OCT is dramatically superior to IVUS. While OCT has been used for coronary artery imaging, previous endovascular OCT devices have not had the physical and biological properties which allowed them to be deployed intracranially with fine precision and control. The disclosed endovascular device accomplishes this with the established safety profile of an intracranial access wire. The structural design and construct our endovascular device uniquely allows intracranial endovascular imaging as well as imaging of extracranial vasculature.

Proposed uses for the illustrated embodiment of the invention can be divided into intracranial and extracranial applications. Intracranial applications include assessment and evaluation of all cerebrovascular diseases including (but not restricted to) atherosclerosis, fibromuscular dysplasia, inflammatory diseases of blood vessels, genetic, chromosomal, developmental, degenerative, and acquired abnormalities such as aneurysms, arteriovenous malformations, dissections, amyloid deposition, blood vessel ruptures and tears, etc, as well as evaluation of therapeutic manipulations such as aneurysm coiling, angioplasty, stent placement, AVM embolizations, tumor embolizations etc. In addition the device can be used for assessment of therapeutic/healing response to surgical and medical treatments as well as prognostication of medical conditions involving blood vessels.

Extracranial applications are the same as intracranial applications but are carried out in blood vessels supplying blood to other organs such as the heart, liver, kidney, extremities, lungs, gastrointestinal tract etc. Two particular uses of great potential are related to cerebral aneurysms. Firstly, by imaging the quantity and quality of fibrous connective tissue including collagen and elastin fibers within the aneurysm wall, the device can be used to predict risk of rupture of cerebral aneurysms. Secondly, by imaging neo-endothelization across the neck of aneurysms treated with coils this device can document the extent of post-treatment aneurysm healing.

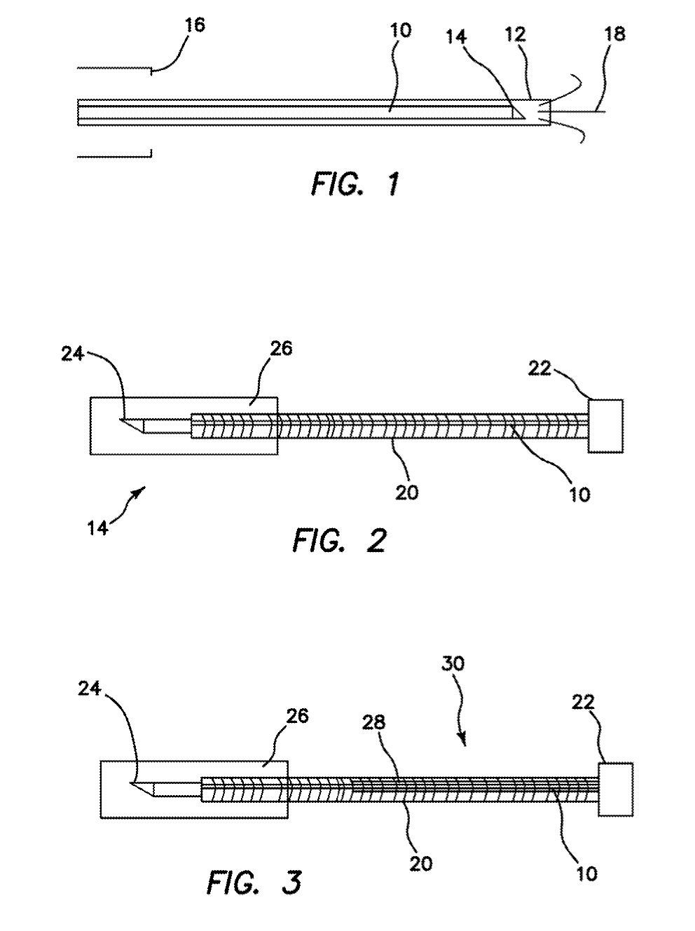

The device is introduced endovascularly from a peripheral blood vessel such as the femoral or brachial artery or vein through an endovascular access sheath 16 as shown in FIG. 1. The device is then passed to its desired location using standard endovascular techniques. Proper positioning of the device adjacent to a region of interest is confirmed using X-ray fluoroscopy. When in desired position the device is rotated, linearly translated, or both through predetermined distances using rotary and linear motors placed outside the body. Concurrently, a bolus flush 18 of 20 cc of normal saline is carried out into the lumen at the region of interest, through an endovascular catheter of appropriate size that is placed two to three centimeters proximal to the imaging field. The saline bolus flushes away luminal blood, which otherwise obstructs the path of light. By a combination of rotary and linear movements three dimensional high-resolution images of blood vessel microstructure can be obtained.

FIG. 1 shows one embodiment for an OCT fiber 10. The OCT fiber 10 is concentrically disposed inside a transparent plastic microcatheter 12. The saline flushes the blood out of the micro catheter 12. OCT fiber 10 rotates and moves linearly telescopes down and up the microcatheter 12. Light from OCT fiber 10 penetrates the transparent microcatheter 12 and reaches the blood vessel wall. The microcatheter 12 allows better translation or linear motion to the tip 14 of the endovascular OCT fiber 10. This embodiment is ideal for imaging proximal portions of blood vessels near an endovascular access site.

FIG. 2 shows the design of endovascular OCT device as a whole apart from microcatheter 12. The core of a conventional guidewire is removed and a single mode optical fiber 10 is inserted into the hollow coil wire 20. In the illustrated embodiment guidewires in two sizes of interest have been investigated. One has a 0.018 inch outer diameter and is ideal for intracranial and coronary vessel imaging by virtue of its small size, while the other has a 0.036 inch outer diameter and is better suited for other vascular sites. In the proximal end, a FC/APC or FC/PC single mode fiber adaptor 22 is assembled. In the distal end, GRIN lens and prism 24 are glued together to direct the light perpendicular to the fiber axis. A glass ferrule 26 is attached with strong medical glue to protect the tip 14 from mechanical damage. This embodiment is better suited for imaging distal vasculature such as cerebral or coronary vasculature.

FIG. 3 shows another embodiment of the endovascular OCT device. In this embodiment a thin steel hollow tube 28 is telescopically disposed over the fiber 10 to increase axial stiffness. This embodiment enables better transfer of rotational torque from the proximal end to the distal tip. Thus, it is to be understood that tube 28 may assume any torsionally stiff element, including but not limited to braided cylinders of metal or stiff polymeric fibers.

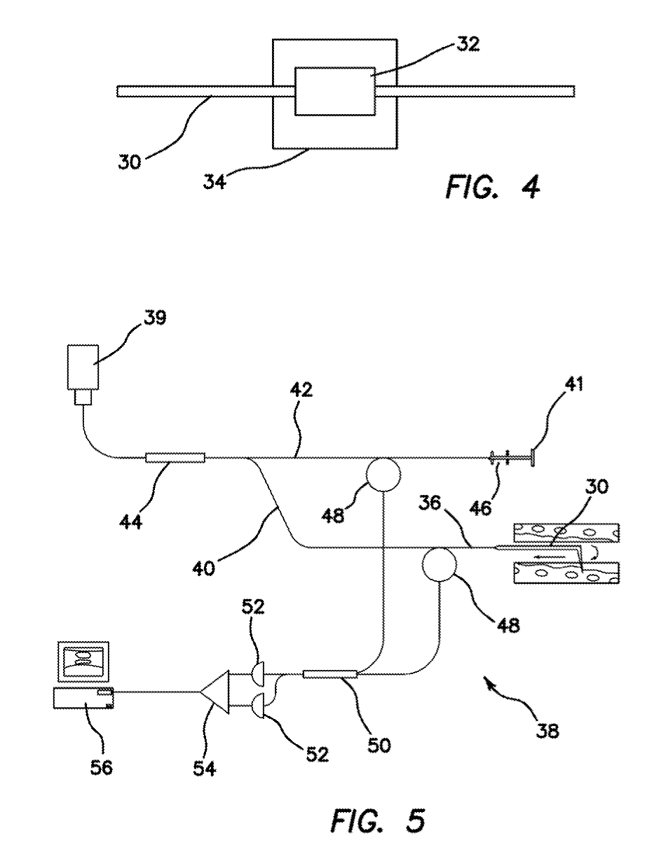

FIG. 4 shows the connection or coupling between OCT probe 30 and the external OCT system. The OCT probe 30 is separately rotated and linearly manipulated by a fiber rotator 32 and linear stage 34, both of which are powered by electric motors that cause mechanical transduction. The combined helix scan mode of OCT probe 30 when both rotator 32 and linear stage 34 are active realizes three dimensional image acquisition. The static optical fiber 36 of FIG. 5 couples the light into OCT probe 30 by the fiber rotator 32.

FIG. 5 shows the schematic diagram of the high speed, high resolution Fourier domain OCT system 38 which reconstructs vessel structure image. 80% of the incident power from swept light source 39 is coupled into sample arm 40 while 20% is feed into reference arm 42 by a 1×2 fiber coupler 44. The reference power directed toward mirror 41 is attenuated by an adjustable neutral density attenuator 46 for maximum sensitivity. Two circulators 48 are used in both reference arm 42 and sample arm 40 to redirect the back-reflected light to the second 2×2 fiber coupler 50 (50/50 split ratio) for balanced detection. The time fringe signal collected by a photodetector 52 is digitized with an analog-digital acquisition card or differential amplifier 54 and transferred to a computer 56 for processing.

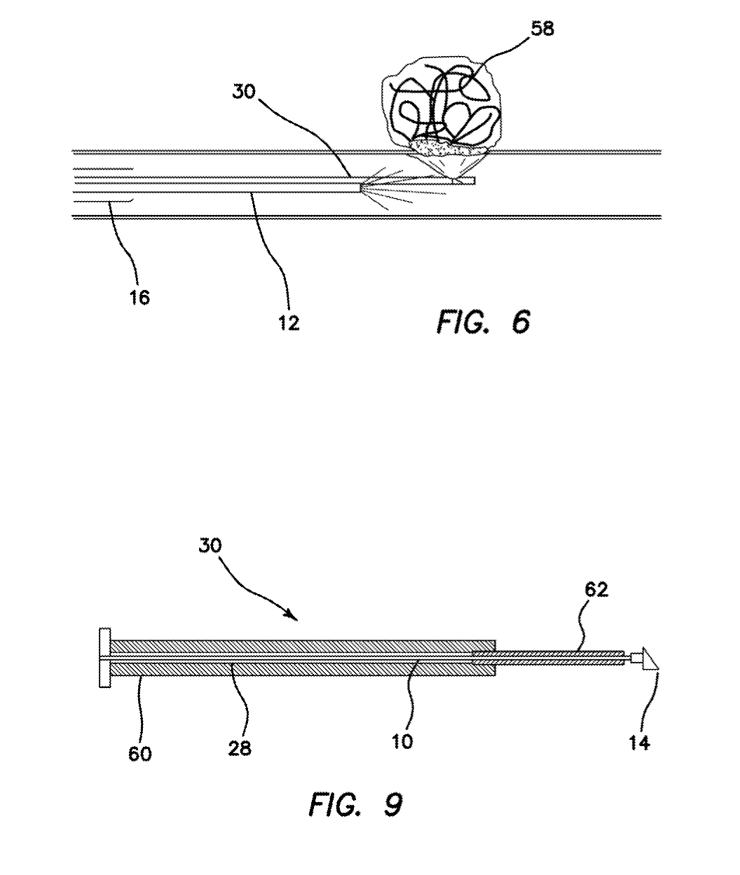

FIG. 6 diagrammatically shows an endovascular application of the OCT probe 30 (labeled as OCT fiber). Probe 30 is used to endovascularly scan a region of interest, such as cerebral aneurysm neck in this case which has been occluded by implanted GDC coils 58. Blood is flushed out using 0.9 normal saline flushes delivered through a microcatheter 12 placed a few centimeters proximal to the region of interest. A larger delivery catheter 16 such as a 6F endocatheter can be used to stabilize the endovascular setup. Linear and rotational scans are obtained using OCT probe pullback and rotation from an external motor (not shown).

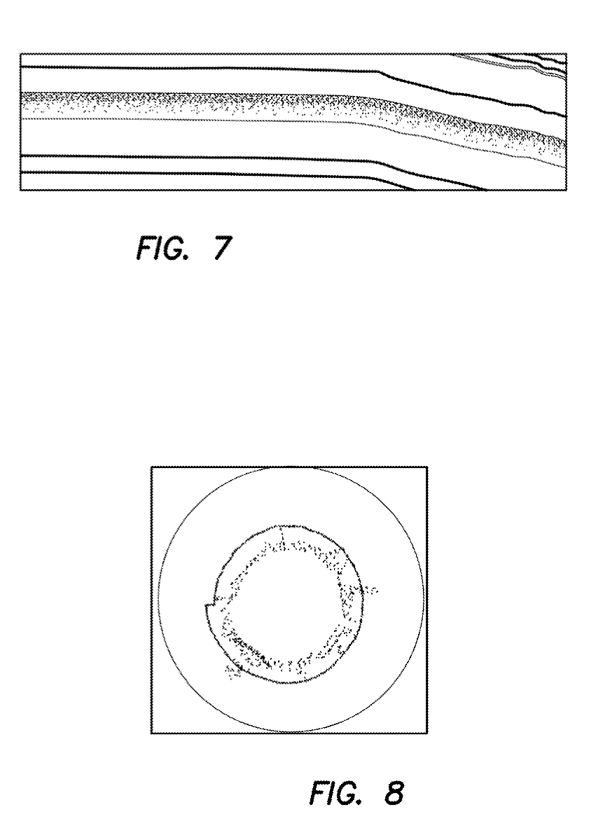

FIGS. 7 and 8 are the first human cerebrovascular OCT images to be recorded, which are made possible from the unique design of our endovascular OCT device. FIG. 7 is the linear scanning OCT image from the cavernous portion of the left Internal carotid artery of a patient. FIG. 8 is a rotational scan obtained from the cavernous portion of the left internal carotid artery of the same patient. Optical shadows of stent struts obtained at the edge of an intracranial stent are can be seen in the images.

Another embodiment includes an OCT brain probe shown in FIG. 9 which uses a two-stage design. The proximal portion of probe 30 is of larger diameter than the distal end. The proximal portion is comprised of an outer coil 60, made by removing the inner core of a larger diameter guide wire (0.036 inch), and an inside hollow steel tube 28. The inside hollow steel tube 28 increases the rigidity of the proximal portion of the entire OCT brain probe 30, which is helpful for transduction of linear movement to the distal tip 14. The distal portion is composed of a smaller coil 62 made by removing the inner core of a smaller diameter guide wire (0.018 inch) and the optical tip 14. The small coil 62 of the distal portion maintains flexibility in the distal portion of the OCT brain probe 30, which is critical for catheter delivery and guidance inside the brain vessel.

It can now be appreciated that the disclosed probe 30 includes several novel and advantageous features . The flush can be performed from the inner annular channel of the probe when it is applied to a mono-channel operation as depicted in FIG. 1. The probe can also be applied in bi-channel operation. In this way, another flushing micro-catheter is applied as depicted in FIG. 6. Each catheter occupies one channel respectively. In the mechanical aspects of the design, the probe tip 14 is secured by a transparent material ferrule, such as glass as depicted in FIG. 3. This feature reduces the probe fracture risk greatly. To adapt to a tortuous human vessel, such as cerebral artery, the probe has a two-part body as depicted in FIG. 9. The proximal portion is relatively large and rigid, while the tip and distal portion is small and soft.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following invention and its various embodiments.

The words used in this specification to describe the invention and its, various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in must be understood as being generic to all possible meanings supported by the specification and by the word itself.

The definitions of the words or elements of the following invention and its various embodiments are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the invention and its various embodiments below or that a single element may be substituted for two or more elements in a claim.

Insubstantial changes from the claimed subject matter as viewed by a person with ordinary skill in the art, now known or later devised, are expressly contemplated as being equivalently within the scope of the invention and its various embodiments. Therefore, obvious substitutions now or later known to one with ordinary skill in the art are defined to be within the scope of the defined elements.

The invention and its various embodiments are thus to be understood to include what is specifically illustrated and described above, what is conceptionally equivalent, what can be obviously substituted and also what essentially incorporates the essential idea of the invention.

We claim:

1. An endovascular OCT probe included in an endovascular access device for intravascular imaging comprising:
a longitudinal lumen defined in the endovascular access device;
a hollow coil wire defining an axial lumen therein;
a single mode optical fiber for transmitting light, the fiber being disposed in the axial lumen of the hollow coil wire so that translation and rotation of the hollow coil wire carrying the optical fiber within the endovascular access device is stabilized for scanning endovascular tissue; and
an optic element for directing light from and into the optical fiber at a distal tip of the optical fiber.

2. The endovascular OCT probe of claim 1 where the endovascular access device has a proximal end and further comprises a FC/APC or FC/PC single mode fiber adaptor assembled thereto.

3. The endovascular OCT probe of claim 1 where the endovascular access probe has a distal end which is used for imaging tissue, and where the optic element comprises:
a GRIN lens and prism disposed on the distal end of the optical fiber to direct light in a beam perpendicular to the longitudinal axis of the fiber; and
a glass ferrule encasing the GRIN lens and prism to protect the optic element and distal tip of the optical fiber from mechanical damage.

4. The endovascular OCT probe of claim 1 further comprising a hollow steel tube disposed within the axial lumen of the hollow coil wire within which tube the optical fiber is disposed, the hollow steel tube being provided in at least a proximal of the probe whereby torsional stiffness of the OCT probe is increased to increase rotational and translational scanning stability of the OCT probe.

5. The endovascular OCT probe of claim 1 further comprising a fiber rotator and a linear stage transducer separately coupled to a proximal end of the OCT probe for provide for independently controlled rotational scanning and translational scanning of images.

6. The endovascular OCT probe of claim 5 further comprising an OCT interferometer and computer coupled thereto for generating images from the returned light from the optical fiber of the probe.

7. The endovascular OCT probe of claim 1 where an annular space is provided between the lumen of the endovascular access device and the hollow coil wire to allow for controlled flushing through a distal end of the OCT probe effective to flush a space between the optic element and tissue being scanned.

8. The endovascular OCT probe of claim 1 further comprising:
a hollow steel tube disposed within the axial lumen of the hollow coil wire within which tube the optical fiber is disposed, the hollow steel tube being provided only in a proximal portion of the probe whereby torsional stiffness of the OCT probe is increased to increase rotational and translational scanning stability of the OCT probe; and
a second more distal hollow coil wire of smaller diameter than the proximal hollow coil, the second distal smaller coil wire including the optical fiber extending from the proximal coil wire and terminating in the optic element, the proximal hollow coil wire being characterized by greater stiffness than the distal hollow coil wire.

9. The endovascular OCT probe of claim 1 further comprising an external OCT system and electric motors coupled to the optical fiber and controlled by the external OCT system, wherein the OCT probe is separately rotated by a fiber rotator and/or linearly translated by a linear stage which are powered by the electric motors controlled by the external OCT system to generate a reproducible image with spatial resolution of at least 5 microns.

10. The endovascular OCT probe of claim 8 where the proximal hollow coil wire is comprised of an outer coil made by removing the inner core of a larger diameter guide wire, and the distal hollow coil wire is comprised of a smaller coil made by removing the inner core of a smaller diameter guide wire.

11. An endovascular OCT probe for intravascular imaging comprising:
a microcatheter with a longitudinal lumen defined therein;
a plurality of hollow coil wires coupled longitudinally to each other to collectively define a longitudinal lumen through the plurality of hollow coil wires, the plurality of hollow coil wires having a maximum stiffness and diameter at a proximal end of the plurality of hollow coil wires and sequencing monotonically with smaller diameters and less stiffness for each of the hollow coil wires until reaching a minimum stiffness and diameter at a distal end of the plurality of hollow coil wires;
an optical fiber for transmitting light being disposed in the longitudinal lumen so that translation and rotation of the hollow coil wire carrying the optical fiber within the endovascular access device is stabilized for scanning endovascular tissue; and
an optic element for directing light from and into the optical fiber at a distal tip of the optical fiber.

12. A method using a stable scanning endovascular OCT probe comprising the step of:
endovascularly rotationally optically scanning tissue and/or endovascularly translationally optically scanning tissue with a stability able to achieve at least 5 micron resolution using the OCT probe in an intracranial or extracranial application, where endovascularly rotationally optically scanning tissue is performed in a torsionally reinforced endovascular OCT probe, and endovascularly translationally optically scanning tissue is performed in a longitudinally reinforced endovascular OCT probe.

13. The method of claim 12 where the intracranial applications include assessment and evaluation of cerebrovascular diseases including but not restricted to atherosclerosis, fibromuscular dysplasia, inflammatory diseases of blood vessels, genetic, chromosomal, developmental, degenerative, and acquired abnormalities such as aneurysms, arteriovenous malformations, dissections, amyloid deposition, blood vessel ruptures and tears, evaluation of therapeutic manipulations such as aneurysm coiling, angioplasty, stent placement, AVM embolizations, tumor embolizations, assessment of therapeutic/healing response to surgical and medical treatments or prognostication of medical conditions involving blood vessels, and where the extracranial applications are the same as intracranial applications but are carried out in blood vessels supplying blood to other organs such as the heart, liver, kidney, extremities, lungs, gastrointestinal tract including cerebral aneurysms by imaging the quantity and quality of fibrous connective tissue including collagen and elastin fibers within the aneurysm wall to predict risk of rupture of cerebral aneurysms, or by imaging neo-endothelization across the neck of aneurysms treated with coils to document the extent of post-treatment aneurysm healing.

14. The method of claim 12 further comprising flushing a bolus of 20 cc of normal saline into an endovascular space of interest through an endovascular catheter of appropriate size that is placed two to three centimeters proximal to the imaging field to flush away luminal blood, which otherwise obstructs the path of light during scanning.

15. The method of claim 12 where endovascularly rotationally optically scanning tissue and/or endovascularly translationally optically scanning tissue with a stability able to achieve at least 5 micron resolution using the OCT probe in an intracranial or extracranial application is solely motor controlled.

16. The method of claim 12 where endovascularly rotationally optically scanning tissue and/or endovascularly translationally optically scanning tissue with a stability able to achieve at least 8 micron resolution using the OCT probe in an intracranial or extracranial application is separately controlled and separately transduced rotation and translation of the OCT probe.

17. The method claim 12 where endovascularly rotationally optically scanning tissue is performed in a torsionally reinforced endovascular OCT probe, and endovascularly translationally optically scanning tissue is performed in a longitudinally reinforced endovascular OCT probe comprised of:
a longitudinal lumen defined in an endovascular access device;
a hollow coil wire defining an axial lumen therein;
a single mode optical fiber for transmitting light, the fiber being disposed in the axial lumen of the hollow coil wire so that translation and rotation of the hollow coil wire carrying the optical fiber within the endovascular access device is stabilized for scanning endovascular tissue; and
an optic element for directing light from and into the optical fiber at a distal tip of the optical fiber.

18. The method claim 12 where endovascularly rotationally optically scanning tissue is performed in a torsionally reinforced endovascular OCT probe having an optical fiber disposed in the lumen of a hollow coil wire and an optic element fixed to a distal end of the optical fiber.

19. The method claim 12 where endovascularly translationally optically scanning tissue is performed in a longitudinally reinforced endovascular OCT probe having an optical fiber disposed in the lumen of a thin solid hollow steel tube and an optic element fixed to a distal end of the optical fiber, the steel tube being disposed with a hollow coil wire.
